(19) United States
(12) Patent Application Publication
(10) Pub. No.: US 2023/0285726 A1
(43) Pub. Date: Sep. 14, 2023

(54) EXTERNAL VENTRICULAR DRAIN PROBE AND ULTRASOUND STYLUS COMPLEX FOR PLACEMENT GUIDED BY CONTINUOUS IMAGING

(71) Applicant: Echopole, Lyon (FR)

(72) Inventors: Marc Lenfant, Dijon (FR); Thibault Portella, Dijon (FR)

(21) Appl. No.: 18/040,711

(22) PCT Filed: Aug. 6, 2021
(86) PCT No.: PCT/FR2021/051452

§ 371 Date: Feb. 6, 2023

(30) Foreign Application Priority Data
Aug. 6, 2020 (FR) FR2008332

(51) Int. Cl. A61M 27/00; A61B 8/00; A61B 8/08

(52) U.S. Cl. CPC A61M 27/006; A61B 8/4488; A61B 8/0841

(57) ABSTRACT

A cerebrospinal fluid drainage system for ventricular draining includes an EVD or VPS probe comprising a flexible tube and a stylus comprising a rigid tube configured to be inserted into the flexible tube. The stylus includes an ultrasound transducer at a distal end of the rigid tube. The rigid tube contains only one or more wires coupled with the ultrasound transducer and does not contain an optical fiber. The ultrasound transducer has an imaging depth extending along an axis of the rigid tube. The distal end of the flexible tube is closed by a deformable tip. The stylus can be temporarily secured to the flexible tube. The system further includes an echograph comprising a screen for displaying an anatomical image calculated in B-mode as a function of signals supplied by the ultrasound transducer.

### CROSS-REFERENCE TO RELATED APPLICATIONS

This application is a national phase entry under 35 U.S.C. § 371 of International Patent Application PCT/FR2021/051452, filed Aug. 6, 2021, designating the United States of America and published as International Patent Publication WO 2022/029395 Al on Feb. 10, 2022, which claims the benefit under Article 8 of the Patent Cooperation Treaty to French Patent Application Serial No. FR2008332, filed Aug. 6, 2020.

### TECHNICAL FIELD

The present disclosure relates to a ventricular drain surgical device intended to drain cerebrospinal fluid transiently (EVD: External Ventricular Drain) or permanently (for example: VPS, Ventriculo-Peritoneal Shunt) that is present in the cranial cavity, using a drain implanted in the ventricles and connected to a drainage system, when the cerebrospinal fluid cannot circulate freely. The present disclosure relates to both the EVD and VPS probes, and their use during the placement step, for treating acute or chronic hydrocephaly and therefore ventricular expansion. The present disclosure is industrially applicable in the medical field and, in particular, in the surgical field.

### BACKGROUND

Hydrocephaly corresponds to an abnormal accumulation of fluid within the ventricular system, increasing the volume of the latter and therefore the intracranial pressure. Ventricular drainage is performed with a catheter so as to control the intracranial pressure by draining cerebrospinal fluid (CSF) and thereby decreasing intracranial pressure.

The EVD probe consists of a ventricular drainage catheter, usually made of silicone, placed over a rigid guidewire that the surgeon uses to implant the EVD probe into the patient's ventricular system. A small hole is drilled through the skull (burr hole) and the EVD probe is inserted through the dura mater and then the brain, transfixing the cerebral parenchyma to the target ventricle.

Freehand placement of the EVD probe requires the neurosurgeon to estimate the three-dimensional position of the target ventricle, usually based on external anatomical landmarks. The ventricle is usually only between 0.5 and 3 cm in diameter and may be located at a viewing depth of between 5 and 50 millimeters, and preferably 4 cm or more. Once the position of the target ventricle is estimated, the EVD catheter is pushed through the brain to the target ventricle. The freehand method provides no way to account for potential irregularities in the patient's anatomy that are not externally apparent. Factors such as intracranial expanding lesions (hematoma, edema, tumor, etc.), residual porencephalic cavities, genetic variability, etc., can also affect the location of the target ventricle.

“Intuitive” placement of a catheter sometimes requires the practitioner to make multiple attempts, resulting in more than one pass through brain tissue to accomplish the required placement of the EVD catheter.

According to the literature, up to 65% of ventricular drain probes placed have a distal end in a non-optimal position (outside the optimal ventricular target). Of these, almost a third are non-functional and require revision and reintegration. Possible complications associated with misplacement of an EVD catheter may include intracerebral hemorrhage, stroke, damage to adjacent brain structures, as well as the need for a repeat operation to replace the misplaced catheter. Higher infection rates are also reported when multiple attempts at EVD placement are required.

Several solutions have been explored, in particular, aiding the positioning of the catheter with an imaging system external to the skull (ultrasound on the surface of the cerebral parenchyma in parallel with the EVD probe requiring a second burr hole, cerebral scanner with placement in stereotactic conditions or guided by virtual reality), the use of a GHAJAR guide to constrain the positioning relative to the surface of the skull, the use of an ultrasound stylus introduced into the drain to provide depth information by linear ultrasound that guides the user to the ventricle.

Known in the state of the art is patent WO2015108917 describing an implantable shunt device for draining cerebrospinal fluid from a subarachnoid space of a patient, which comprises a shunt having first and second opposite ends, the second end being adapted to penetrate a wall of a sigmoid, transverse, right or sagittal sinus of the patient, a check valve, a hollow passage extending between the second end and the check valve such that cerebrospinal fluid can be drained from the second end and expelled through the valve, and a mechanism coupled to the shunt and adapted to anchor the shunt at a desired location near the subarachnoid space.

Also known is the article Dual Orientation 16-MHz Single-Element Ultrasound Needle Transducers for Image-Guided Neurosurgical Intervention (authors Yun Jiang, Zhen Qiu, et al.) School of Engineering & Physical Sciences Institute of Sensors, Signals & Systems Institute of Mechanical, Process & Energy Engineering Energy Academy. This article concerns the incorporation of an ultrasound device into the type of biopsy needles commonly used as an interventional tool to provide feedback to neurosurgeons during surgical procedures. To identify the most appropriate path to access a targeted tissue site, single-element transducers that face forward or sideways have been used.

In a different field, which is that of spinal punctures allowing a sample of cerebrospinal fluid to be taken from time to time not in the cranial cavity, but at the spine, patent application WO2017192603 is known, which relates to a needle delivery system comprising a needle and an ultrasound transducer element attached to the distal end of the needle. The system also comprises a needle constraint assembly configured to receive and constrain the needle only to rotational degrees of freedom within a range of angular motion. The system also comprises an ultrasound data processor configured to communicate with the transducer element to receive ultrasound detection signals and to communicate with the needle sensor system to receive needle angular orientation signals.

This solution of the prior art relates to procedures performed by emergency physicians or neurologists to sample cerebrospinal fluid (CSF), a vital fluid in the diagnosis of many diseases and conditions of the central nervous system (CNS). To perform this procedure, a doctor palpates the lower back and identifies the L3 through L5 vertebrae. Once identified, the doctor proceeds to apply a local anesthetic before inserting and advancing a needle, usually a Quincke needle.

This solution is not suitable for an operation to implant a drain in the ventricles intended to be connected to a drainage system, when the cerebrospinal fluid cannot circulate freely.

The solutions of the prior art are not fully satisfactory because they only provide depth information in a one-dimensional mode exploring only a single line, which is the axial line of fire of the ultrasound probe. This information only partially helps the practitioner on the relevance of the guidance and the orientation of the probe. They are designed to minimize cross-section to allow passage through thin drainage catheters less than 1.7 mm in cross-section.

To exploit the information provided by the solutions of the prior art, the practitioner must learn to interpret it, which may require a significant amount of time before he is able to interpret it immediately, reflexively, during a procedure.

### BRIEF SUMMARY

The present disclosure aims to overcome the drawbacks of the state of the art by providing a cerebrospinal fluid drainage system comprising an EVD probe and an ultrasound stylus insertable into the EVD probe, characterized in that the ultrasound stylus comprises a rigid tube provided at its distal end with a scanning transducer forming a beam whose axis is directed along the longitudinal axis of the ultrasound stylus.

In its broadest sense, the present disclosure relates to a cerebrospinal fluid drainage system comprising an EVD or VPS probe.

The probe comprises a flexible tube with an outer diameter of between 1.9 and 5 millimeters and a stylus fitted at its distal end with an ultrasound transducer, which can be inserted into the tube. The system includes an echograph comprising a display screen with an image calculated as a function of the signals provided by the ultrasound probe. The system is characterized in that:
the stylus comprises a rigid tube with an outer diameter corresponding to the inner diameter of the tube containing only the supply wires of the transducer, excluding an optical fiber, the rigid tube provided at its distal end with a transducer of a diameter identical to the diameter of the rigid tube and having an imaging depth in the axis of the rigid tube between 0 and 100 millimeters,
the distal end of the tube is closed by a solid and deformable tip , and
the proximal end of the tube (FIG. 6) has a securing system with the proximal end of the ultrasound probe. This securing system has five (5) objectives: 1) to limit the longitudinal impaction of the ultrasound stylus in the VD tube during its insertion, 2) to restrict the rotation between the tube (10) and the ultrasound stylus, 3) to fix the depth of insertion of the stylus in the tube, 4) to create a secured system between the ultrasound stylus and the drain in order to prevent mobility of one or the other of the two elements of the assembly, and 5) to generate a fluid space distal to the ultrasound probe that is conducive to its use.

The securing system may be of the screw, bayonet, anchor, Velcro or notch type, this list being non-limiting. In a variant, the proximal end of the tube bearing the securing system is reinforced and detachable from its functional portion. The removable portion may be pre-punched or can be sectioned intraoperatively using a cutting device such as a scalpel or medical scissors. Favorably, this detachable portion will be transparent in order to be able to visually confirm the correct attachment of the assembly. In another variant, the fixing portion is a screw thread (Huer-lock type) and is non-removable, facilitating and securing its connection to the collection tubing and its pocket (FIG. 6), thus making it possible to limit mismatches compared to a conventional interlocking system.

In a variant, the tube has one or more internal lateral bores of greater longitudinal axis complementary to the central bore. Some or all of these bores respond to the complementary shape of the ultrasound stylus (in the form of segmental fins or longitudinal blades) in order to maximize their joining and to limit the degree of rotation distal to the assembly. Alternatively, one or more of these bores will advantageously make it possible to instill, from the proximal end of the assembly, a trans-echogenic fluid such as physiological serum at the interface of the piezoelectric elements of the ultrasound stylus and of the inner face of the tip of the VD tube.

The section of the tube is round in shape. Alternatively, the section of the tube will be asymmetrical, thickened next to the lateral internal bores in order to maintain satisfactory resistance to bending after removal of the ultrasound stylus (FIG. 6).

The ultrasound stylus and possibly the VD tube present a proximal visual marker making it possible to know the orientation of the ultrasound probe and to facilitate the spatial location of the operator during the intra-parenchymal insertion of the assembly.

The system provides B-mode ultrasound imaging.

According to a variant, the echograph provides B-mode ultrasound imaging associated with at least one other imaging modality comprising color mode, pulsed Doppler, power and 3D mode.

Advantageously, the transducer is a scanning transducer.

According to a variant, the system further comprises a sterile enclosure surrounding the stylus and whose end in which the transducer is engaged contains an ultrasound gel.

According to another variant, the distal bottom of the tube has a shape complementary to the external shape of the transducer.

According to a first embodiment, the tip has a conical shape. According to another embodiment, the tip has a hemispherical/alternative shape with the aim of limiting the generation of optical aberrations by the deflection of the ultrasound beams that it generates (ultimately responsible for ultrasound artifacts).

The tip and/or the whole of the EVD tube are made of biocompatible materials with biomechanical properties similar to those usually expected for a drain probe and, advantageously, with a material having the lowest possible coefficient of attenuation of the ultrasound beam. The thickness of this tip may advantageously be reduced for the same purpose.

Advantageously, the tip contains an ultrasound gel or instilled sterile water. This instillation may be done before the ultrasound scanner-tube assembly by prior immersion of the VD tube in a basin of physiological serum or by its instillation via the internal lumen of the tube. In a variant, the assembly will be fixed and the anterograde installation will be done by the lateral internal bores, or by direct puncture using a needle from the conical tip of the VD tube.

According to a variant, the system further comprises a computer executing a computer program for preprocessing the signal supplied by the scanning transducer, the preprocessing consisting in applying compensation for variations in the propagation conditions in the tip of the tube.

Preferably, the working frequency of the transducer is between 4 Mhz and 15 Mhz, more advantageously 10 Mhz.

According to a variant, the tip of the probe comprises an echogenic marker.

### BRIEF DESCRIPTION OF THE DRAWINGS

Other features and advantages of the present disclosure will become clear upon reading the following detailed embodiments, with reference to the accompanying figures.

FIG. 1 is a sectional view of a system according to the present disclosure.

FIG. 2 is a view of the end of the probe according to a first embodiment.

FIG. 3 is a view of the end of the probe according to a second embodiment.

FIG. 4 is a sectional view of the end of the probe according to a third embodiment.

FIG. 5 is a sectional view of the end of the probe according to a fourth embodiment.

FIG. 6A is a view of the stylus according to a first position.

FIG. 6B is a view of the stylus according to a second position.

FIG. 6C is a view of the stylus according to a third position.

FIG. 7A is a sectional view of a first variant of the geometry of the tube according to the present disclosure.

FIG. 7B is a sectional view of a second variant of the geometry of the tube according to the present disclosure.

FIG. 7C is a sectional view of a third variant of the geometry of the tube according to the present disclosure.

FIG. 7D is a sectional view of a fourth variant of the geometry of the tube according to the present disclosure.

FIG. 7E is a cross-sectional view of a first variant of the tube according to the present disclosure.

FIG. 7F is a cross-sectional view of a first variant of the tube according to the present disclosure.

FIG. 8A is a sectional view of the tube according to the present disclosure with a solid tip.

FIG. 8B is a sectional view of the tube according to the present disclosure with a solid tip after introduction of the trans-echogenic fluid.

FIG. 8C is a sectional view of the tube according to the present disclosure with a solid tip after introduction of the fluid and the ultrasound stylus.

FIG. 8D is a sectional view of the tube according to the present disclosure with a hollow tip.

FIG. 8E is a sectional view of the tube according to the present disclosure with a hollow tip after introduction of the trans-echogenic fluid.

FIG. 8F is a sectional view of the tube according to the present disclosure with a hollow tip after introduction of the ultrasound stylus, then introduction of the fluid through the groove.

FIG. 8G is a sectional view of the tube according to the present disclosure with a hollow tip after introduction of the fluid and the ultrasound stylus.

FIG. 9 is a sectional view of the tube according to the present disclosure.

FIG. 10 is a sectional view of the ultrasound stylus.

FIG. 11 is a sectional view of the system formed by the stylus engaged in the ventricular drain tube.

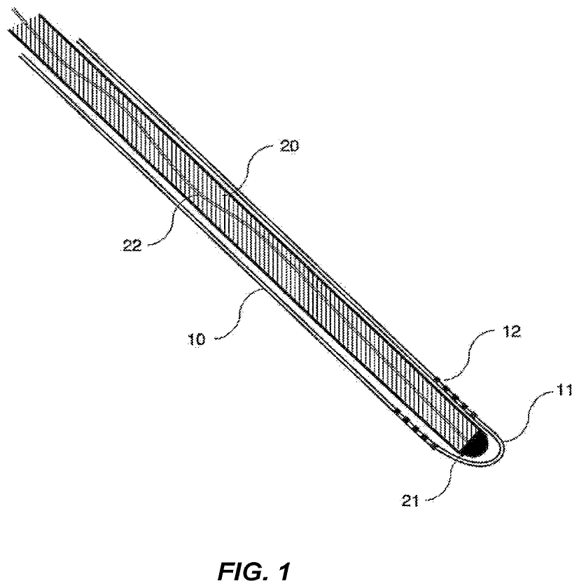

### DETAILED DESCRIPTION

The present disclosure relates to a system comprising an EVD or VPS probe constituting a drainage tube (10) and an ultrasound stylus (20) for guided implantation.

The outer diameter of the drainage tube (10) is typically between 1.9 millimeters and 5 millimeters, and the inner diameter from 1.7 millimeters to 3.5 millimeters. The outer diameter of the stylus (20) is typically 0.5 millimeters less than the inner diameter of the drainage tube, with a median channel for the passage of electrical wires connecting the transducer to the ultrasound system.

The thickness of the tip (11) at the distal end of the tube (10) is between 1 and 7 mm depending on the power of the ultrasound probe and the shape of the tip. This tip (11) is solid, or more favorably, it has an ellipsoidal or quadrangular central bore fitting the surface of the Doppler stylus. In an assembly, this bore is maximal, of ellipsoidal, conical, pyramidal or quadrangular shape or even of a similar shape, in order to accommodate the Doppler stylus-flexible enclosure complex. The tip (11) may, for example, be silicone, a silicone-based material, that is to say, comprising at least 10% silicone, rubber, plastic, polyamide, polyether block amide, polycarbonate, polyimide or polytetrafluoroethylene, this list being non-exhaustive.

The drainage tube (10) is perforated distally and laterally with multiple openings (12) approximately 2 mm in diameter over a span of approximately 20 mm. These openings (12) can be pores, holes, orifices or slots and pass all the way through the membrane of the tube (10). These openings (12) are preferably of a size suitable for the passage of water, cerebrospinal fluid and blood. The pores, holes, orifices or slots may be adapted according to their ability to allow the cerebrospinal fluid to flow, with variable shapes that may be cylindrical, conical or oval, this list not being exhaustive. The distance between the openings (12) is variable, ranging from 1 to 4 mm to allow the proper flow of cerebrospinal fluid.

The end of the tuber (10) forms a conical or pyramidal or ellipsoidal tip (11) and is non-rigid. This tip is solid with a proximal internal bore in order to match the shape of the distal end of the ultrasound probe, but may have an internal bore of a shape suitable for receiving a flexible enclosure-Doppler stylus complex or a fluid interface.

The flexible drainage tube (10) is intended to be implanted for a short or long term in a patient in order to drain the ventricular system in the event of acute or chronic hydrocephaly. This tube (10) is connected to a gravity drain system using a collection bag once it is in place in case of EVD and to a drainage system connected to the peritoneum or to the vascular system in case of VPS.

In case of VPS, the flexible drainage tube has the same characteristics in its distal portion (tip, holes, pores or drainage orifices). The intracranial length is 4 to 9 cm with a right angle allowing the application of the tube to the cranial cavity using a reservoir, continued by a drainage tube that will be connected to the bypass valve. The junction area between the drain and the ultrasound stylus can be cut in order to be connected to the valve.

The channel of this drainage tube (10) makes it possible to accommodate an ultrasound stylus (20) formed by a removable rigid mandrel whose length exceeds the length of the drainage tube (10), to facilitate the introduction of the device into the cerebral parenchyma and to facilitate its removal after placement of the EVD/VPS probe. This ultrasound stylus comprises a scanning ultrasound transducer (21) powered by wires (22). This is, for example, an ALOKA UST 534 brand transducer. This ultrasound stylus is inserted into the drainage tube along the bore area using the fin and its final position is locked by the proximal fixing system in order to secure the assembly and generate a distal fluid space that is conducive to the operation of the ultrasound probe. The introduction of the assembly will be oriented according to the marker of the ultrasound stylus indicating the orientation of the ultrasound beam and its anterior part.

The active element of the transducer consists mainly of a piezoelectric material, optionally piezocomposite, optionally multilayer, and of a set of at least two electrodes that make it possible to create an electric field in the thickness of the piezoelectric material. Preferably, one or more acoustic adaptation layers are integrated into this active element, on the front face of the active element, to facilitate the acoustic transfer toward the front of the transducer.

The transducer is advantageously glued to the distal end of the stylus (20) by an epoxy glue.

This transducer may consist of a network of piezoelectric micro-transducers, or of a single transducer driven by an oscillating movement by a MEMS-type support. It delivers a 2D or 3D signal of the environment in front of the conical tip (11) in order to allow the operator to guide the advancement and the orientation of the rigid mandrel during the installation of the drain through the cerebral parenchyma of a patient. The signals are used by a 2D ultrasound scanner in B mode to provide an image of the area in front of the probe.

The working frequency is typically 10 Mhz. The transducer (21) is connected to an echograph.

This ultrasound device has a “B” ultrasound mode (2D anatomical imaging), possibly accompanied by a “C” mode (color), a “D” mode (Doppler), a power mode and/or a 3D mode, with an imaging depth between 0 and 50-100 millimeters. The ultrasound probe is preferably of the linear, curvilinear or micro-convex type, but may correspond to any type of ultrasound probe making it possible to obtain a B-type ultrasound image along the longitudinal axis of the stylus (suitable IVUS-type microprobe (intravascular), trapezoidal probe, etc.).

The signal supplied by the transducer can be corrected, in the event of inhomogeneity of the tip (11), by a computer applying digital processing to compensate for variations in transmission induced by the material constituting the tip (11) and variations in thickness depending on the angle of the shot.

### TIP CONFIGURATION

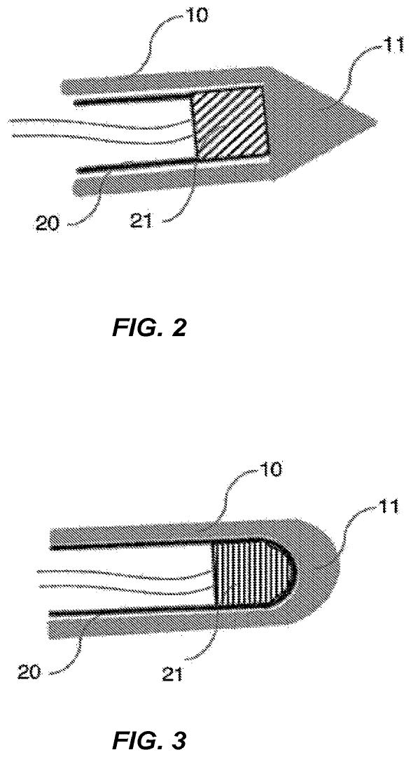

FIGS. 2 to 5 illustrate different configurations of the tip (11) of the stylus (20). It may be conical as shown in FIG. 2 or hemispherical as shown in FIG. 3.

The distal end of the ventricular drain is produced in so as to be as atraumatic as possible, in a biocompatible material that is as trans-echogenic as possible.

Its thickness and geometric conformation will aim to reduce optical aberration artefacts and attenuation of the ultrasound beam.

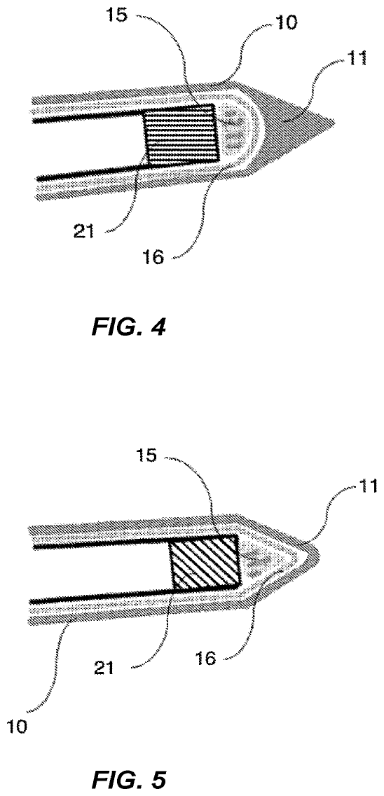

It may also receive an ultrasound gel (15) housed between the probe (21) and the inside of the tip (11) as shown in FIG. 4 or 5.

The sterile ultrasound gel is attached to the tip of the ultrasound stylus by a sterile flexible enclosure (16), producing a flexible enclosure-Doppler stylus complex that is loaded within the drain probe. In this variant, the inner diameter of the EVD probe is slightly greater than the outer diameter of the flexible enclosure-Doppler stylus complex (FIG. 2). The length of the flexible enclosure is such that once unrolled on the Doppler stylus, it exceeds the surface of the cranial cavity at all times and remains less than the length of the Doppler stylus (approximately between 6 and 20 cm).

Alternatively, a trans-echogenic fluid such as saline is directly instilled at the transducer-tip interface of the drain tube. This instillation can be carried out before assembly of the tube-ultrasound stylus complex, or after assembly by means of an internal bore of the tube complementary to the central bore receiving the stylus, or by direct puncture using a fine needle from a space at the posterior face of the tip of the tube. Thus, in these variants, the length of the portion of the ultrasound stylus inside the drain tube will be slightly less than the internal bore of the tube in order to produce this fluid space that is conducive to the use of the ultrasound probe.

Optionally, the tip of the probe comprises a lateral echogenic marker whose purpose is to form a visible reference on the transcranial ultrasound image.

### CONFIGURATION OF THE PROXIMAL PART

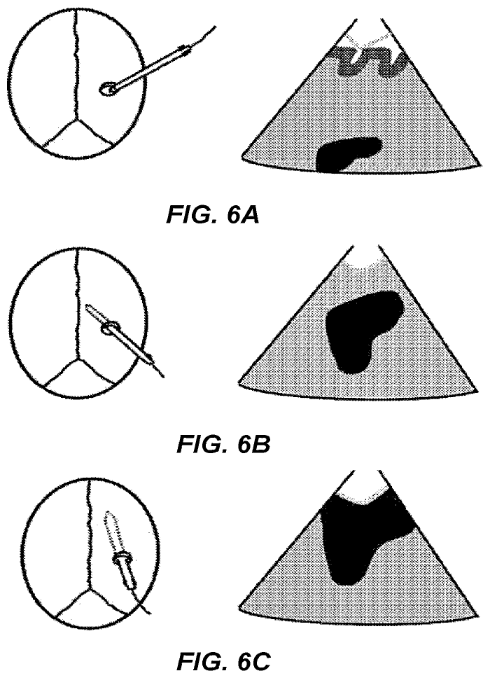

FIG. 6 illustrates the male and female screw pitch between the ultrasound probe and the drain tube making it possible to obtain a connection between the two components. Once locked, the thread makes it possible 1) to limit the longitudinal impaction of the VD tube during its insertion, 2) to restrict the rotation between the tube (10) and the ultrasound stylus, 3) to fix the insertion depth of the stylus into the tube, 4) to create a system that is secured between the ultrasound stylus and the drain in order to prevent mobility of one or the other of the two elements of the assembly, and 5) to generate a fluid space distal to the ultrasound probe that is conducive to its use. This last point ensures that there is a tight fluid space at the tube/ultrasound interface regardless of the movements imparted to the assembly at the distal end of the assembly (FIG. 6).

Alternatively, the thread of the drain tube allows its connection to the collection tubing and to the pocket (X), limiting the risks of mismatching with respect to a conventional interlocking system. In another variant, the fixing system is present on the most proximal segment of the tube and is removable intraoperatively.

### CONFIGURATION OF THE FRONT TUBE PART

FIGS. 6A to 6C illustrate the front part of the ellipsoidal tube with a thicker front part with a groove in its internal part allowing the fin of the ultrasound probe to be received in order to avoid incorrect paths and to secure the introduction.

At its outer anterior part is a marking indicating the orientation of the ultrasound probe and the direction of introduction within the cerebral parenchyma (FIG. 6).

### IMPLEMENTATION OF THE PRESENT DISCLOSURE

A punctate skin opening is made using anatomical landmarks in front of the coronal suture 2 or 3 cm laterally from the midline. Then a burr hole about 5 mm to 3 cm in diameter is made, with opening and sometimes coagulation of the dura mater.

Instillation of sterile water with a syringe along the internal bore of the drain to produce a distal fluid collection suitable for use of the ultrasound probe.

The ultrasound stylus (20) is engaged in the central internal bore of the tube (10) of the VD probe to stiffen it, by inserting the fin of the ultrasound probe into the lateral internal bore of the ventricular drain to avoid any misdirection until locking by the proximal fixation system. In the final assembly, the ultrasound transducer is embedded in the tip of the ventricular drain tube with a fluid interface guaranteed by the prior instillation of saline. The assembly makes it possible to acquire information on the environment of the tip of the tube (10) and to orient the operator's gestures. The locking of the screw pitch is done before inserting the assembly in contact with the cerebral parenchyma and is done by visual control of the markers on the surface of the ultrasound stylus and of the tube, as well as by direct visual control in a variant. The operator places the tip of the VD probe on the surface of the cerebral parenchyma and visualizes the ventricular target. Usually, it corresponds to the frontal horn of an ipsilateral lateral ventricle in the context of an EVD, and in the ipsilateral ventricular junction for VPS. Once spotted, the operator gradually pushes the complex through the cerebral parenchyma under continuous ultrasound guidance until the target is reached.

The ultrasound stylus (20) is then withdrawn from the tube (10), by unscrewing the proximal thread and sliding the fin in the internal bore of the drain, freeing the lumen of the EVD drain and allowing immediate reflux of CSF by its proximal end. The suction mechanism may be based on gravity alone. To this end, the fluid collection pocket must be located below the head in order to obtain a siphon effect.

At the end opposite the perforated deformable tip (11), a suction system fitted with a connection endpiece to a tubing (Micro Vac type, Elite Surgical Supplies) is screwed into the EVD probe, in order to allow a secure connection reducing the risk of maladjustment and reducing the risk of infection inherent in this complication. The depression generated is adapted to the clinical context, generally 1 bar. In the case of a VPS, the drainage system is connected to a drainage valve regulating the flow and the whole is continued by the abdominal catheter located either in the peritoneum or in the vascular system at the vena cava.

The volume of CSF that is thus extracted from the cranial cavity makes it possible to control and treat severe intracranial hypertension, thus avoiding the multiple complications that it induces.

If performing a ventriculo-peritoneal shunt (VPS) or a ventriculo-atrial shunt (VAS), a retro-auricular skin incision is made; a 2 to 3 cm burr hole is made. The dura mater is coagulated and then incised. The ultrasound stylus is loaded into the VPS probe. The operator places the tip of the ultrasound stylus complex inserted in the VPS probe on the surface of the cerebral parenchyma and visualizes the ventricular target. Usually, it corresponds to the ventricular junction. Once spotted, the operator gradually pushes the complex through the cerebral parenchyma under continuous ultrasound guidance until the target is reached. The ultrasound stylus is then withdrawn, freeing the lumen of the VPS catheter and allowing immediate reflux of CSF through its proximal end. The assembly will be connected to a bypass valve and an abdominal or atrial catheter.

### TUBE OUTER DIAMETER

For drainage applications without intraventricular bleeding, a tube having an outer diameter of 1.5 to 1.9 millimeters will preferably be chosen.

For drainage applications with intraventricular bleeding, a tube having an outer diameter of 2.5 to 5 millimeters, and preferably less than 4 millimeters, will preferably be chosen.

### TUBE TIP CONFIGURATION

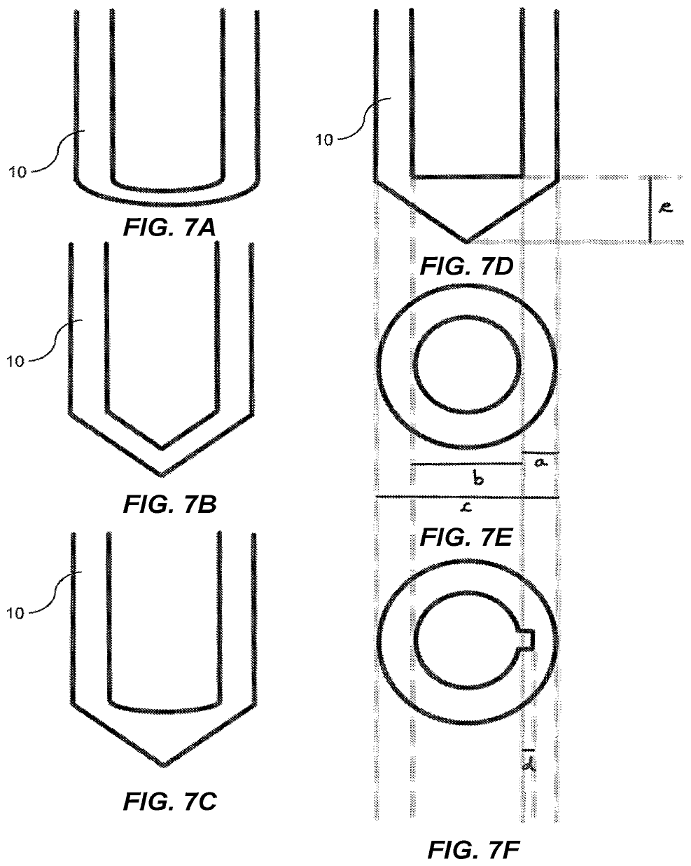

FIGS. 7A to 7G illustrate different embodiments of the deformable tip of the tube (10). It may present:
A slightly concave end with a frontal wall of substantially constant thickness (FIG. 7A) to form a domed hollow frontal end;
A tapered end with a front wall of substantially constant thickness (FIG. 7B) to form a hollow pointed end; or
A tapered end with a planar transverse bottom (FIG. 7C) to form a solid pointed end.

FIG. 7D illustrates the height a of the solid and deformable tip, between 0.7 mm and 7 mm.

The wall thickness n of the tube (10) is about 0.7 mm and the internal bore is between 1.9 and 3 mm (FIG. 7E).

The outer diameter of the tube (10) is between 1.9 and 4 mm.

FIG. 7F illustrates a variant where the wall of the tube (10) has a groove (17) forming a bore with a depth less than the thickness of the wall, i.e., less than 0.7 mm. Optionally, the wall thickness is increased at the groove.

This bore makes it possible to inject a trans-echogenic fluid into the bottom of the tube (10).

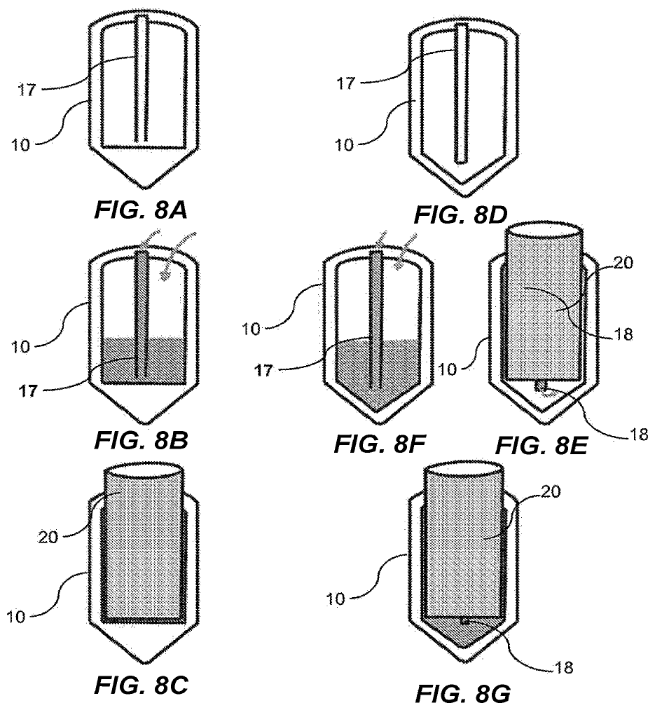

FIGS. 8A to 8C illustrate the tube (10) with a solid and deformable tip, and optionally a groove for injecting a trans-echogenic fluid before the introduction of the stylus carrying an ultrasound transducer at its distal end. This fluid may be introduced via the lateral bore (17) or via the central bore.

FIGS. 8D to 8G illustrate the tube (10) with a hollow and deformable tip, and optionally (FIG. 7G) a groove (17) to inject a trans-echogenic fluid under the stylus carrying an ultrasound transducer at its distal end, the circulation being performed through the internal lumen (18) (FIG. 8E).

Instillation of trans-echogenic fluid

According to a first embodiment, the fluid is introduced into the tube before the ultrasound stylus is inserted, so that the probe comes into contact with the fluid filling the internal volume of the hollow tip.

According to a second embodiment, the ultrasound stylus is introduced into the tube, and then fluid is injected into the tube through a longitudinal bore (17) formed in the wall of the tube (10).

According to a third variant, the ultrasound stylus is introduced into the tube and then trans-echogenic fluid is injected into the interior volume of the tip of the probe by means of a syringe, the needle of which passes through the wall of the tube (10).

Ventricular drain system

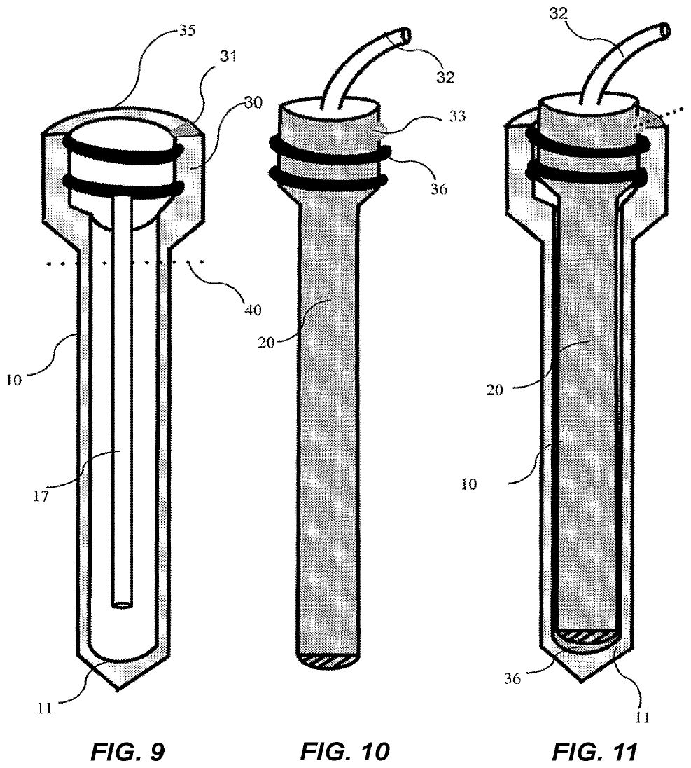

FIGS. 9 to 11 illustrate the present disclosure and present the ventricular drain probe alone (FIG. 9), the ultrasound stylus alone (FIG. 10) and the stylus engaged in the ventricular drain probe (FIG. 11).

The probe (10) has a section (30) with a thicker wall on the side of the opening for introducing the stylus (20) and/or made of a material that is more rigid than the material of the probe (10). The edge of the open end of the probe has a visual marker (31) to provide an angular reference. The section (30) has an internal thread (35) for fixing the stylus (20) having a complementary thread (35). This thread (35) also makes it possible to then fix a fluid drainage pocket.

The probe (10) has a weakening groove (40) optionally allowing the upstream part of the probe (10) to be severed.

The stylus (20) also has a visual marker (33) allowing the angular orientation of the stylus with respect to the probe (10) and its correct angular alignment, as well as the direction of observation, to be checked.

The bottom of the tip (11) contains trans-echogenic fluid (36) providing the interface between the ultrasound probe and the drain probe (10). The stylus (20) protrudes slightly from the thickened section (30) of the drain tube (10).

According to a variant, the parietal thickening is asymmetrical.

1.-14. (canceled)

15. A cerebrospinal fluid drainage system for ventricular draining, comprising:
an EVD or VPS probe comprising a flexible tube; and
a stylus comprising a rigid tube configured to be inserted into the flexible tube, the stylus including an ultrasound transducer at a distal end of the rigid tube, a diameter of the rigid tube corresponding to an inner diameter of the flexible tube, the rigid tube containing only one or more wires coupled with the ultrasound transducer and not containing an optical fiber, the ultrasound transducer having a diameter at least substantially identical to the diameter of said the rigid tube, the ultrasound transducer having an imaging depth extending along an axis of the rigid tube, the stylus comprising a feature located and configured to couple the stylus to the flexible tube, the distal end of the flexible tube being closed by a deformable tip; and
an echograph comprising a screen for displaying an anatomical image calculated in B-mode as a function of signals supplied by the ultrasound transducer.

16. The system of claim 15, wherein the deformable tip is hollow to form a pocket configured to receive a trans-echogenic fluid therein.

17. The system of claim 15, wherein the feature located and configured to couple the stylus to the flexible tube comprises a recess, and wherein the flexible tube has a thread configured to interact with the recess for temporary engagement of the flexible tube with the stylus.

18. The system of claim 15, wherein the echograph provides B-mode ultrasound imaging associated with at least one other imaging modality selected from among a color mode, a pulsed Doppler, a power mode, or a 3D mode.

19. The system of claim 15, wherein the transducer is a scanning transducer.

20. The system of claim 15, further comprising a sterile enclosure surrounding the stylus, an end of the sterile enclosure adjacent the ultrasound transducer containing an ultrasound gel.

21. The system of claim 15, wherein the distal end of the rigid tube has a shape complementary to a shape of the ultrasound transducer.

22. The system of claim 15, wherein the deformable tip of the stylus has a conical shape.

23. The system of claim 15, wherein the deformable tip of the stylus has a hemispherical shape.

24. The system of claim 15, further comprising a computer executing a computer program for preprocessing the signals supplied by the ultrasound transducer, the preprocessing comprising applying compensation for variations in propagation conditions in the deformable tip of the stylus.

25. The system of claim 15, wherein the deformable tip of the stylus comprises an echogenic marker.

26. The system of claim 15, further comprising a securing mechanism configured to secure the stylus and the flexible tube together to produce a stable fluid space at a distal end of the stylus.

27. The system of claim 15, further comprising drainage system including a drainage tube and a collection bag, a proximal end of the flexible tube comprising a connector for joining the flexible tube to the drainage tube of the drainage system.
